(19) United States
(12) Patent Application Publication
(10) Pub. No.: US 2026/0174389 A1
(43) Pub. Date: Jun. 25, 2026

(54) HOME PHOTOTHERAPEUTIC RECTAL TREATMENT

(71) Applicant: PHOTOPILL MEDICAL LTD., Rehovot (IL)

(72) Inventors: Sharon BEN YEHUDA, Rehovot (IL); Ram BEN YEHUDA, Rehovot (IL)

(21) Appl. No.: 19/125,760

(22) PCT Filed: Nov. 2, 2023
(86) PCT No.: PCT/IL2023/051127

§ 371 Date: Apr. 30, 2025

Related U.S. Application Data
(60) Provisional application No. 63/382,186, filed on Nov. 3, 2022.

(51) Int. Cl. A61B 5/00; A61B 5/1455

(52) U.S. Cl. CPC A61B 5/6826; A61B 5/0084; A61B 5/14552

(57) ABSTRACT

Rectal phototherapy device comprising a treatment probe, light emitters, pressure sensors, proximity sensors, controller, and user interface. Probe includes cylindrical probe housing and is inserted into a rectal cavity. Emitters are disposed in probe housing and emit light through housing toward a treatment area of rectal cavity. Pressure sensors are disposed in probe housing and detect a pressure exerted by rectal wall on treatment probe. Proximity sensors are disposed in probe housing, such as at distal end of housing, and detect a proximity of probe to rectal wall. User interface issues alert responsive to a threshold pressure of detected pressure and responsive to a threshold proximity of detected proximity. Controller controls light emission of light emitters in accordance with phototherapeutic protocols determined based on detected proximity and predefined phototherapeutic treatment plan. Probe housing may be single flexible element or plurality of housing segments flexibly linked together.

### FIELD OF THE INVENTION

The present invention relates to the field of phototherapeutic treatment, and particularly to self-administrable phototherapeutic treatment devices.

### BACKGROUND OF THE INVENTION

Phototherapeutic treatment has proven to be beneficial in the treatment of various physical conditions and disorders, including diseases of the gastrointestinal (GI) tract. In certain situations, patients lack access to doctors and other medical personnel to apply the phototherapy and must therefore self-administer the phototherapy rectally. There are dangers associated with self-deployment of a rectal phototherapeutic device in that a patient could inadvertently damage the rectal wall during self-deployment. Accordingly, there is a need for a safe rectal phototherapeutic device facilitating self-deployment.

### SUMMARY OF THE INVENTION

In accordance with one aspect of the present invention, there is thus provided rectal phototherapy device. The device includes a treatment probe including a cylindrical probe housing, the treatment probe operable for insertion into a rectal cavity. The device includes a plurality of light emitters, disposed in the probe housing, each of the light emitters configured to emit light through the probe housing toward a treatment area of the rectal cavity. The device includes a plurality of pressure sensors, disposed in the probe housing, each of the pressure sensors configured to detect a pressure exerted by a rectal wall on the treatment probe. The device further includes a controller, communicatively coupled with the light emitters and the pressure sensors, the controller configured to control a light emission of the light emitters. The device further includes a user interface, communicatively coupled with the controller, the user interface operative to issue an alert responsive to a threshold pressure of the pressure detected by the pressure sensors. The device may further include a plurality of proximity sensors, disposed in the probe housing, each of the proximity sensors configured to detect a proximity of the treatment probe to the rectal wall. The proximity sensors may be circumferentially disposed at a distal end of the probe housing. The user interface may be operative to issue an alert responsive to a threshold proximity of the proximity detected by the proximity sensors. The controller may be configured to control light emission characteristics of the emitted light in accordance with phototherapeutic protocols determined based on a proximity detected by the proximity sensors and based on a treatment plan of a phototherapeutic treatment. The phototherapeutic protocols may include: light emission characteristics; a pulsed beam emission sequencing; a continuous beam emission sequencing; an emitted light wavelength; an emitted light intensity; an emitted light duration; an emitted light pulse width; an emitted light pulse rate; and/or a treatment dose of the phototherapeutic treatment. The cylindrical probe housing may be implemented as a single flexible element. The cylindrical probe housing may be implemented as a plurality of housing segments, each of the housing segments flexibly linked to another of the housing segments. The light emitters may be configured to radially emit light through circumferentially arranged transparent windows of the treatment probe. A wavelength of the emitted light may be in the range of: 440 nm-600 nm; 600 nm-700 nm; 700 nm-1000 nm; or 1000 nm-1500 nm. An intensity of the emitted light may be in the range of: 0.1 J/cm²-3.0 J/cm²; 3.1 J/cm²-9.0 J/cm²; and 9.1/cm²-12.0 J/cm². At least one of the light emitters may include: a laser; a light emitting diode (LED); and/or a fluorescent lamp. At least one the pressure sensors may include: a piezoelectric sensor, an electromagnetic pressure sensor, and/or a capacitive pressure sensor. At least one of the proximity sensors may include: a photoelectric proximity sensor; an ultrasonic proximity sensor; an inductive proximity sensor; and/or a capacitive proximity sensor. The user interface may include at least one of: an audible indicator; a visual indicator; and/or a tactile indicator. The emitted light may be directed for a treatment of proctitis.

In accordance with another aspect of the present invention, there is thus provided a method for administration of rectal phototherapy. The method includes the step of inserting a treatment probe of a rectal phototherapy device into a rectal cavity, the treatment probe comprising a cylindrical probe housing. The method includes the step of detecting a pressure exerted by a rectal wall on the treatment probe, by a plurality of pressure sensors disposed in the probe housing. The method includes the step of issuing an alert responsive to a threshold pressure of the pressure detected by the pressure sensors. The method includes the step of emitting light through the probe housing toward a treatment area of the rectal cavity, by a plurality of light emitters, disposed in the probe housing. The method may further include the step of detecting a proximity of the treatment probe to the rectal wall, by a plurality of proximity sensors disposed in the probe housing. The method may further include the step of issuing an alert responsive to a threshold proximity of the proximity detected by the proximity sensors. Light emission characteristics of the emitted light may be controlled in accordance with phototherapeutic protocols determined based on a proximity detected by the proximity sensors and based on a treatment plan of a phototherapeutic treatment. The phototherapeutic protocols may include: light emission characteristics; a pulsed beam emission sequencing; a continuous beam emission sequencing; an emitted light wavelength; an emitted light intensity; an emitted light duration; an emitted light pulse width; an emitted light pulse rate; and/or a treatment dose of the phototherapeutic treatment. The light emitted by the light emitters may be radially emitted through circumferentially arranged transparent windows of the treatment probe. A wavelength of the emitted light may be in the range of: 440 nm-600 nm; 600 nm-700 nm; 700 nm-1000 nm; or 1000 nm-1500 nm. An intensity of the emitted light may be in the range of: 0.1 J/cm²-3.0 J/cm²; 3.1 J/cm²-9.0 J/cm²; and 9.1/cm²-12.0 J/cm². The emitted light may be directed for a treatment of proctitis.

### BRIEF DESCRIPTION OF THE DRAWINGS

The present invention will be understood and appreciated more fully from the following detailed description taken in conjunction with the drawings in which:

FIG. 1 is a schematic side view illustration of a rectal phototherapy device disposed in a rectal cavity, constructed and operative in accordance with an embodiment of the present disclosure;

FIG. 2 is a schematic side view illustration of a segmented treatment probe of a rectal phototherapy device disposed in a rectal cavity, constructed and operative in accordance with another embodiment of the present disclosure; and

FIG. 3 is a general flow diagram of a method for administration of rectal phototherapy, operative in accordance with an embodiment of the present disclosure.

It will be appreciated that for the sake of clarity, elements shown in the figures may not be drawn to scale and reference numerals may be repeated in different figures to indicate corresponding or analogous elements.

### DETAILED DESCRIPTION OF THE EMBODIMENTS

In the following detailed description, specific details are set forth in order to facilitate understanding of the invention; however, it should be understood by those skilled in the art that the present invention may be practiced without these specific details.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and claims and should not be interpreted in an idealized or overly formal sense unless expressly so defined herein. Well-known functions or constructions may not be described in detail for brevity and/or clarity.

It will be understood that, although the terms first, second, etc., may be used herein to describe various elements, components, regions, layers and/or sections, these elements, components, regions, layers and/or sections should not be limited by these terms. Rather, these terms are only used to distinguish one element, component, region, layer and/or section, from another element, component, region, layer and/or section.

It will be understood that when an element is referred to as being “on”, “attached” to, “operatively coupled” to, “operatively linked” to, “operatively engaged” with, “connected” to, “coupled” with, “contacting”, “added to”, etc., another element, it can be directly on, attached to, connected to, operatively coupled to, operatively engaged with, coupled with, added to, and/or contacting the other element or intervening elements can also be present. In contrast, when an element is referred to as being “directly contacting” another element or “directly added” to another element, there are no intervening elements and/or steps present.

Whenever the terms “about” or “approximately” are used, it is meant to refer to a measurable value such as an amount, a temporal duration, and the like, and is meant to encompass variations from the specified value, as such variations are appropriate to perform the disclosed methods.

Certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Whenever terms “plurality” and “a plurality” are used it is meant to include, for example, “multiple” or “two or more”. The terms “plurality” or “a plurality” may be used throughout the specification to describe two or more components, devices, elements, units, parameters, or the like. The term set when used herein may include one or more items. Unless explicitly stated, the method embodiments described herein are not constrained to a particular order or sequence. Additionally, some of the described method embodiments or elements thereof can occur or be performed simultaneously, at the same point in time, or concurrently.

This disclosure employs open-ended permissive language, indicating for example, that some embodiments “may” employ, involve, or include specific features. The use of the term “may” and other open-ended terminology is intended to indicate that although not every embodiment may employ the specific disclosed feature, at least one embodiment employs the specific disclosed feature.

The terms “user” and “operator” are used interchangeably herein to refer to any individual person or group of persons using or operating a device or method according to one or more embodiments of the present invention.

The terms “subject” and “patient” are used interchangeably herein to refer to an individual upon which a device or method according to one or more embodiments of the present invention is operated upon, such as a person upon which a phototherapy procedure (e.g., a rectal phototherapy procedure) is performed. The subject may be any living entity, such as a human, animal, or other vertebrate. It is noted that a “user” and a “patient” are not necessarily mutually exclusive, such that a user may self-administer a device according to a disclosed embodiment.

The term “phototherapy”, also known as “light therapy”, is used herein to broadly encompass all forms of treatment based on the application of light to a body part, such as for treating one or more physical conditions or medical disorders, including but not limited to ultraviolet (UV) light and other types of light emissions having other wavelength and/or characteristics.

The present disclosure will be apparent from the following detailed description, which proceeds with reference to the accompanying drawings, wherein the same references relate to the same elements. For a better understanding of certain embodiments and to show how the same may be carried into effect, reference will now be made, purely by way of example, to the accompanying drawings in which like numerals designate corresponding elements or sections throughout.

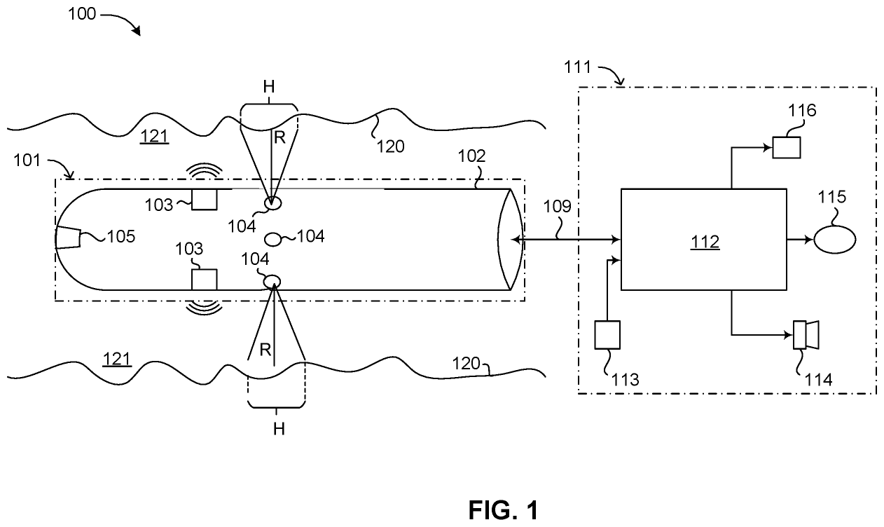

Reference is made to FIG. 1, which is a schematic side view illustration of a rectal phototherapy device, generally referenced 100, disposed in a rectal cavity, constructed and operative in accordance with an embodiment of the present invention. Rectal phototherapy device 100 includes a treatment probe 101, operable to be inserted into a rectal cavity of a patient, such as via self-deployment and in a home setting. Treatment probe 101 is shown disposed in between rectal walls 120 of a rectal cavity 121. Treatment probe 101 is in communication with a controller unit 111 disposed outside rectal cavity 121. Treatment probe 101 includes a cylindrical housing 102. Housing 102 includes a plurality of proximity sensors 103 circumferentially spaced around housing 102. Proximity sensor 103 is configured for detecting a proximity of treatment probe 101 to rectal wall 120. Housing 102 further includes a plurality of circumferentially spaced light emitters 104, configured to emit therapeutic light for administration of phototherapy of a rectal wall 120 of rectal cavity 121. Additionally, housing 102 includes at least one pressure sensor 105 abutting a distal end thereof. Pressure sensor 105 is configured to detect a pressure level at rectal wall 120, i.e., a pressure level exerted by rectal wall 120 on probe 101. Treatment probe 101 may be fitted with at least one proximity sensor 103 at a distal end thereof.

Emitters 104 may emit light through a circumferential series of transparent windows through which the emitted light radiates and expands as a function of distance from its source. As shown, a tissue treatment area A is calculated according to the following equation: 2×π×H×R; where “H” represents the height of the beam in reference to the length of probe 101, and “R” represents the radius of rectum cavity 121 measured from the emitter 104.

Controller unit 111 is communicatively coupled with treatment probe 101, such as via a wired or wireless communication channel. Control unit 111 includes a processer (not shown), for receiving and providing information or instructions to/from other components of device 100 and performs requisite data processing. Control unit 111 includes a memory (not shown), for storing information required by device 100, such as sensor data, treatment protocols, and processing algorithms for modulating phototherapy responsively to sensor data. Control unit 111 includes a controller 112, configured to selectively control the operation of device 100, and may dynamically adjust operational parameters of components thereof. For example, controller 112 may modulate the light emission of emitters 104, such as by implementing a sequential stopping and restarting light emission scheme, and/or by modifying one or more properties of the emitted light, such as a frequency or intensity thereof, in accordance with a relevant phototherapy treatment protocol. Control unit 111 includes a power source 113, for powering controller 112 and optionally other components of device 100. Control unit 111 includes a user interface, which may be embodied by at least one of: an audible indicator 114, a visual indicator 115, and a tactile indicator 116, and configured for providing an indication or feedback to a user of device 100 relating to the operation thereof, such as an alert. Audible indicator 114 may be a speaker, for providing an audible alert (e.g., a beeping sound or voice command). Visual indicator 115, may be a light source, for providing a visual alert (e.g., via flashing lighting). Tactile indicator 116 may be a vibrating device, for providing a tactile alert. For example, a received alert or feedback from the user interface may be utilized to direct a user when inserting treatment probe 100. Proximity sensors 103, pressure sensors 105, and light emitters may be powered by power source 113 or by a different power source (not shown) such as a battery contained within housing 102 or control unit 111.

Device 100 may optionally include and/or be associated with additional components not shown in the Figures for enabling the implementation of the disclosed subject matter. For example, device 100 may include a protective covering for covering treatment probe 101 when not in use and for removing prior to operation.

Housing 102 of treatment probe 101 may be implemented from semi-flexible polymeric material, such as polycarbonates, specifically Makrolon® polycarbonate. Other suitable polymeric materials may include: acrylic, high-density polyethylene, polyetherimide, polypropylene, and the like. Such materials can become flexible enough to avoid tissue damage at low thickness, such as 0.1 mm-0.4 mm, to deform when pressed against rectal walls 120 at a high pressure, for example at a pressure exceeding 1 kg/cm². In other embodiments, flexible or semi-flexible materials may be used to construct a probe housing 102 having only a flexible tip while the rest of housing 102 remains rigid. Typically, probe housing 102 may be constructed through injection molding.

Pressure sensor 102 may be implemented, for example, as a piezoelectric sensor, an electromagnetic pressure sensor, a capacitive pressure sensor, and the like.

Proximity sensor 103 may be implemented, for example, as a photoelectric proximity sensor, an ultrasonic proximity sensor, an inductive proximity sensor, a capacitive proximity sensor, and the like.

Light emitter 104 may be implemented, for example, as a laser, a light emitting diode (LED), a fluorescent lamp, and the like. Emission wavelengths of emitted light may range, for example, between 440 to 600 nanometers (nm); between 600 nm-700 nm, between 700 nm-1000 nm, and between 1000 nm-1500 nm. Light intensities of emitted light may range, for example, between 0.1 J/cm²-3.0 J/cm², between 3.1 J/cm²-9.0 J/cm², and between 9.1/cm²-12.0 J/cm².

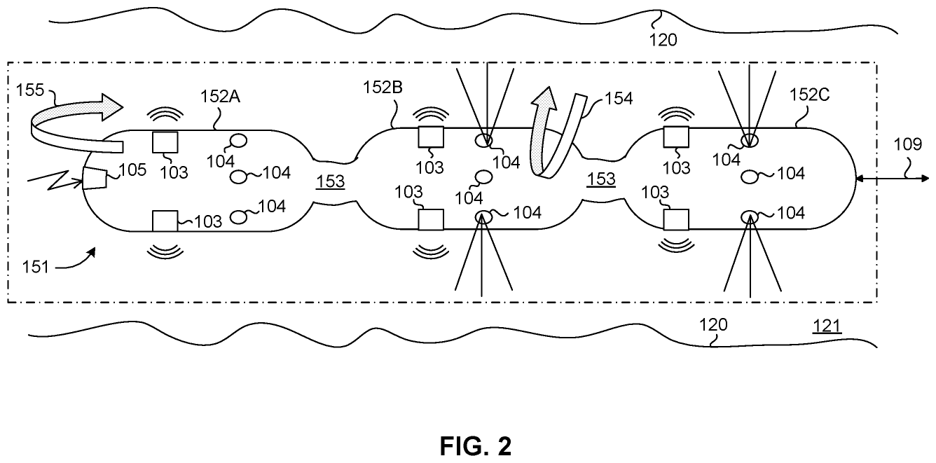

Reference is made to FIG. 2, which is a schematic side view illustration of a segmented treatment probe, referenced 150, of a rectal phototherapy device disposed in a rectal cavity, constructed and operative in accordance with another embodiment of the present disclosure. Treatment probe 151 is shown disposed in between rectal walls 120 of a rectal cavity 121. Treatment probe 151 is generally analogous to treatment probe 101 (FIG. 1) but is implemented with a segmented housing having a plurality of housing segments 152A, 152B, 152C. Each of housing segments 152A, 152B, 152C is linked to an adjacent one of housing segments 152A, 152B, 152C through a flexible joint 153, for enabling treatment probe 150 to buckle at joints 153 during insertion, such as when a distal end of a housing segment 152A, 152B, 152C contacts a portion of rectal wall 120. Such buckling of treatment probe 150 may function as a safety feature to enable a user who is self-administering device 100 (e.g., at a home setting) to insert treatment probe 150 into rectal cavity 121 while avoiding puncturing or damaging a portion of rectal wall 120, such as by a distal end of housing segment 152A. This potential hazard may result from a protrusion of rectal wall 120 into the pathway of rectal cavity 121 and/or by a curvature of rectal cavity 121. As shown, at least some of housing segments 152A, 152B may be rotatable in at least one direction along a rotation axis, as depicted by arrows 154, 155. Housing segments 152A, 152B, 152C may be implemented from a flexible material, such as a rubber or polymeric rubber compound, to provide flexibility thereof.

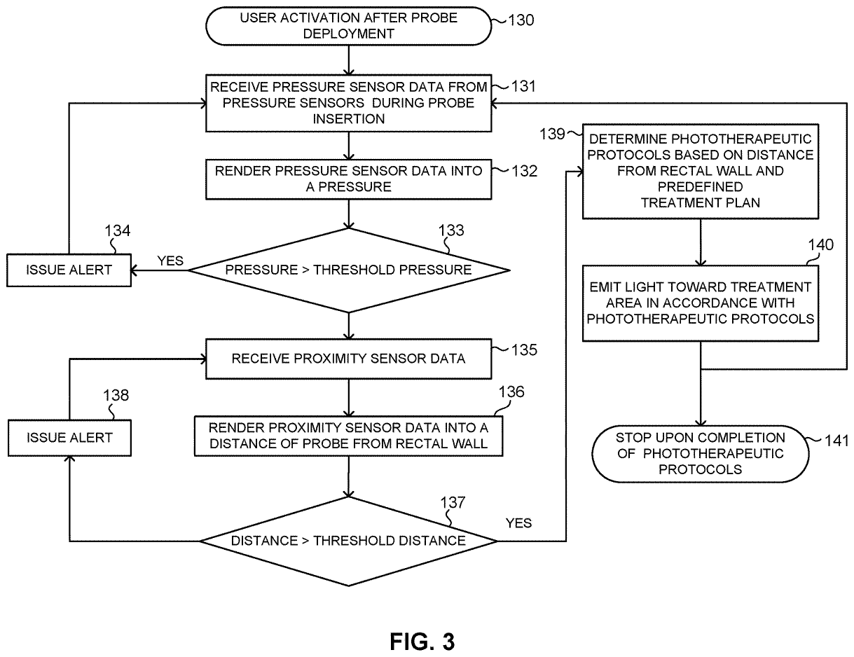

Reference is now made to FIG. 3, which is a general flow diagram of a method for administration of rectal phototherapy. The method may be self-administered by a user, such as in a non-clinical or home setting. The method includes processing steps for facilitating patient safety and dose optimization for a rectal phototherapeutic treatment of the user. The processing steps will be described in view of FIG. 1, by way of example. Processing commences after probe deployment by a user at step 130. The user inserts treatment probe 101 into rectal cavity 121 of the user at a desired insertion depth. The user may decide when a proper insertion depth has been achieved, such as based on a physical sensation of the probe advancement. Alternatively, an insertion depth of treatment probe 101 may be ascertained from sensor readings obtained by proximity sensors 103, such as based on predetermined changes of rectal cavity diameter as markers of insertion depth. In step 131, pressure sensors receive pressure sensor data during probe insertion. Pressure sensors 105 obtain pressure readings upon contact of a distal end of treatment probe 101 with a rectal wall 120 of rectal cavity 121. The pressure reading of a respective pressure sensor 105 reflects a pressure level exerted by rectal wall 120 on probe 101. In a next step 132, pressure sensor data is rendered into a pressure value. The pressure readings obtained by pressure sensors 105 may be rendered into a pressure value for subsequent processing in accordance with the type of pressure sensor 105 employed. In a next step 133, the pressure value is compared with a threshold pressure. A detected pressure value, rendered from the pressure readings of pressure sensor 105, is compared with a predetermined threshold pressure value stored in a memory of control unit 111, where the threshold pressure value is indicative of a maximum pressure that can be tolerated by rectal wall 120 without damage. If the detected pressure value exceeds the threshold pressure value, then an alert is issued in step 134. Referring to FIG. 1, a user interface of control unit 111 issues an alert or warning signal indicative of a danger pressure level, such as via audible indicator 114, visual indicator 115, and/or tactile indicator 116. After issuance of the alert, processing continues in step 131 by continuing to obtain pressure sensor data to ensure treatment probe is safely deployed. When the pressure value is within acceptable limits (i.e., below the threshold pressure value), processing continues in step 135 in which proximity sensor data is received. Proximity sensors 103 of treatment probe 101 obtain proximity readings respective of a proximity of treatment probe 101 to rectal wall 120. In a next step 136, proximity sensor data is rendered into a proximity value. The proximity readings obtained by proximity sensors 103 may be rendered into a proximity value respective of a distance from rectal wall, for subsequent processing, in accordance with the type of proximity sensor 103 employed. In a next step 137, the proximity value is compared with a threshold proximity. A detected proximity value, rendered from the proximity readings of proximity sensor 103, is compared with a predetermined threshold proximity value stored in a memory of control unit 111, where the threshold proximity value is indicative of a maximum proximity or minimum distance to rectal wall 120 for avoiding damage thereto. If the detected proximity value exceeds the threshold proximity value, then an alert is issued in step 138. Referring to FIG. 1, a user interface of control unit 111 issues an alert or warning signal indicative of a danger proximity level, such as via audible indicator 114, visual indicator 115, and/or tactile indicator 116. After issuance of the alert, processing continues in step 135 by continuing to obtain proximity sensor data to ensure treatment probe is safely deployed. It is noted that the pressure sensing and associated user feedback (e.g., steps 131, 132, 133, 134) and the proximity sensing and associated user feedback (e.g., steps 135, 136, 137, 138) may be performed in parallel (e.g., concurrently). When the proximity value (i.e., distance from rectal wall 120) is within acceptable limits (i.e., below the threshold proximity value), processing continues in step 139 in which phototherapeutic protocols are determined based on the proximity value and a predefined treatment plan. A processor of control unit 111 determines phototherapeutic protocols for a phototherapeutic treatment of user based on the detected proximity value (i.e., distance of treatment probe 101 from rectal wall 120), and in accordance with a predefined treatment plan associated with the phototherapeutic treatment (e.g., stored in a memory of control unit 111). The phototherapeutic protocols may include light emission characteristics, such as emission sequencing (e.g., pulsed on continuous beam); wavelength; intensity; duration; pulse width; pulse rate. The light emission characteristics may be based on a maximum or minimum distance to rectal wall 120 (e.g., according to a threshold proximity value) and/or a maximum insertion depth along rectal cavity 121, such as based on predefined data. The phototherapeutic protocols may further include at least one treatment dose. For example a treatment dose (expressed in Joule per square centimeters), may be defined according to the following formula: Power (Watts) x Time (sec.)/treatment area (cm²). In a next step 140, light is emitted toward the treatment area in accordance with the determined phototherapeutic protocols. Emitters 102 of treatment probe 101 emit light toward a treatment area, such as at a portion of a rectal wall 120 of rectal cavity 121 in accordance with the phototherapeutic protocols, for implementing a phototherapeutic treatment of the user. Controller 112 may control the light emission characteristics of the light emitted by emitters 120 in accordance with the phototherapeutic protocols. Optionally, the processing may return to step 131 after a selected duration, such as during one or more stages of the phototherapeutic treatment, to receive updated pressure sensor data and proximity sensor data for ensuring that treatment probe 101 is maintained safely positioned within rectal cavity 121. In step 141, the processing ends upon completion of the phototherapeutic treatments, such as following a prescribed time duration or power delivery of the phototherapeutic protocols.

It is appreciated that the rectal phototherapy device according to disclosed embodiments may be self-administered or self-deployed by a user. The disclosed device and method may be used for treatment of physical conditions or disorders of the gastrointestinal (GI) tract, such as proctitis in various forms, causes and degrees of severity. The disclosed device and method may allow for phototherapeutic treatment in a non-clinical setting, such as at home, rather than at a dedicated medical facility or treatment clinic. The disclosed device and method may be self-administered by an ordinary user without requiring prior experience or skills, and without requiring assistance from a qualified medical practitioner or clinician. The disclosed device and method incorporates a variety of safety features facilitating safe and effective self-deployment, such as a multisensory alert system, including distal pressure sensors operative to detect contact of the probe with the rectal wall, proximity sensors operative to detect proximity to the rectal wall, indicators operative to provide an alert if a threshold pressure or threshold proximity is reached. The safety features may also include a flexible housing of the treatment probe configured to buckle under a pressure sufficient to damage a rectal wall.

While certain embodiments of the disclosed subject matter have been described, so as to enable one of skill in the art to practice the present invention, the preceding description is intended to be exemplary only. It should not be used to limit the scope of the disclosed subject matter, which should be determined by reference to the following claims.

1. A rectal phototherapy device, comprising:
a treatment probe, comprising a cylindrical probe housing, the treatment probe operable for insertion into a rectal cavity;
a plurality of light emitters, disposed in the probe housing, each of the light emitters configured to emit light through the probe housing toward a treatment area of the rectal cavity;
a plurality of pressure sensors, disposed in the probe housing, each of the pressure sensors configured to detect a pressure exerted by a rectal wall on the treatment probe;
a controller, communicatively coupled with the light emitters and the pressure sensors, the controller configured to control a light emission of the light emitters; and
a user interface, communicatively coupled with the controller, the user interface operative to issue an alert responsive to a threshold pressure of the pressure detected by the pressure sensors.

2. The rectal phototherapy device of claim 1, further comprising a plurality of proximity sensors, disposed in the probe housing, each of the proximity sensors configured to detect a proximity of the treatment probe to the rectal wall.

3. The rectal phototherapy device of claim 2, wherein the proximity sensors are circumferentially disposed at a distal end of the probe housing.

4. The rectal phototherapy device of claim 2, wherein the user interface is operative to issue an alert responsive to a threshold proximity of the proximity detected by the proximity sensors.

5. The rectal phototherapy device of claim 3, wherein the controller is configured to control light emission characteristics of the emitted light in accordance with phototherapeutic protocols determined based on a proximity detected by the proximity sensors and based on a treatment plan of a phototherapeutic treatment.

6. The rectal phototherapy device of claim 5, wherein the phototherapeutic protocols comprises at least one protocol selected from the group consisting of: light emission characteristics; a pulsed beam emission sequencing; a continuous beam emission sequencing; an emitted light wavelength; an emitted light intensity; an emitted light duration; an emitted light pulse width; an emitted light pulse rate; and a treatment dose of the phototherapeutic treatment.

7. The rectal phototherapy device of claim 1, wherein the cylindrical probe housing comprises a single flexible element.

8. The rectal phototherapy device of claim 1, wherein the cylindrical probe housing comprises a plurality of flexibly linked housing segments.

9. The rectal phototherapy device of claim 1, wherein the light emitters are configured to radially emit light through circumferentially arranged transparent windows of the treatment probe.

10. The rectal phototherapy device of claim 1, wherein a wavelength of the emitted light is selected from the group consisting of: 440 nm-600 nm; 600 nm-700 nm; 700 nm-1000 nm; and 1000 nm-1500 nm.

11. The rectal phototherapy device of claim 1, wherein an intensity of the emitted light is selected from the group consisting of: 0.1 J/cm²-3.0 J/cm²; 3.1 J/cm²-9.0 J/cm²; and 9.1/cm²-12.0 J/cm².

12-16. (canceled)

17. A method for administration of rectal phototherapy, the method comprising the steps of:
inserting a treatment probe of a rectal phototherapy device into a rectal cavity, the treatment probe comprising a cylindrical probe housing;
detecting a pressure exerted by a rectal wall on the treatment probe, by a plurality of pressure sensors disposed in the probe housing;
issuing an alert responsive to a threshold pressure of the pressure detected by the pressure sensors; and
emitting light through the probe housing toward a treatment area of the rectal cavity, by a plurality of light emitters, disposed in the probe housing.

18. The method of claim 17, further comprising the step of detecting a proximity of the treatment probe to the rectal wall, by a plurality of proximity sensors disposed in the probe housing.

19. The method of claim 18, further comprising the step of issuing an alert responsive to a threshold proximity of the proximity detected by the proximity sensors.

20. The method of claim 17, wherein light emission characteristics of the emitted light is controlled in accordance with phototherapeutic protocols determined based on a proximity detected by the proximity sensors and based on a treatment plan of a phototherapeutic treatment.

21. The method of claim 20, wherein the phototherapeutic protocols comprises at least one protocol selected from the group consisting of: light emission characteristics; a pulsed beam emission sequencing; a continuous beam emission sequencing; an emitted light wavelength; an emitted light intensity; an emitted light duration; an emitted light pulse width; an emitted light pulse rate; and a treatment dose of the phototherapeutic treatment.

22. The method of claim 17, wherein the light emitted by the light emitters is radially emitted through circumferentially arranged transparent windows of the treatment probe.

23. The method of claim 17, wherein a wavelength of the emitted light is selected from the group consisting of: 440 nm-600 nm; 600 nm-700 nm; 700 nm-1000 nm; and 1000 nm-1500 nm.

24. The method of claim 17, wherein an intensity of the emitted light is selected from the group consisting of: 0.1 J/cm²-3.0 J/cm²; 3.1 J/cm²-9.0 J/cm²; and 9.1/cm²-12.0 J/cm².

25. (canceled)
